(19) United States
(12) Patent Application Publication
(10) Pub. No.: US 2023/0201036 A1
(43) Pub. Date: Jun. 29, 2023

(54) UV-LASER-BASED SYSTEM FOR CORRECTING VISION DISORDERS

(71) Applicant: Carl Zeiss Meditec AG, Jena (DE)

(72) Inventors: Hartmut Vogelsang, Oberweser (OT Arenborn) (DE); Beate Böhme, Großpürschütz (DE)

(21) Appl. No.: 17/999,548

(22) PCT Filed: May 21, 2021
(86) PCT No.: PCT/EP2021/063609

§ 371 Date: Nov. 21, 2022

(30) Foreign Application Priority Data
May 24, 2020 (DE) 10 2020 206 423.7
Jul. 10, 2020 (DE) 10 2020 208 676.1

(51) Int. Cl. A61F 9/008

(52) U.S. Cl. CPC A61F 9/00814; A61F 9/00804; A61F 2009/00872; A61F 2009/00878; A61F 2009/00897

(57) ABSTRACT

A focusing optical system for a UVL-LVC system with a UV laser source and a scanning system that focuses a laser in a focal field and a lens assembly with a convergent focal field. The invention further includes a planning unit that generates planning data for a UVL-LNC system with a UV laser source, a scanning system, a focusing optical system, and a control unit for controlling the UVL-LVC system while taking into consideration planning data, wherein the planning unit takes into consideration geometry losses, Fresnel losses, and/or a spatial extension of laser radiation on a working surface while calculating the planning data, and the planning unit has an interface that provides the planning data. Finally, the invention includes a UVL-LVC system with a UV laser source, a scanning system, a focusing optical system according to the invention, a planning unit according to the invention, and a control unit.

### RELATED APPLICATIONS

This application is a National Phase entry of PCT Application No. PCT/EP2021/063609 filed May 21, 2021, which application claims the benefit of priority to DE Application No. 10 2020 208 676.1 filed Jul. 10, 2020, and DE Application No. 10 2020 206 423.7 filed May 24, 2020, the entire disclosures of which are incorporated herein by reference.

### TECHNICAL FIELD

Example embodiments of the invention relate to a focusing optical unit for a UV laser-based system for vision correction (UVL-LVC system) having a UV laser source for providing laser radiation and a scanning system for lateral scanning of the laser radiation in the x- and y-directions. An example embodiment of the invention furthermore relates to a planning unit for generating planning data for a UV laser-based system for vision correction having a UV laser source for providing laser radiation, a scanning system for lateral scanning of the laser radiation in the x- and y-directions, a focusing optical unit for directing the laser radiation to a work surface and a control unit designed to control the UVL-LVC system taking the planning data into consideration. Finally, an example embodiment of the present invention relates to a UV laser-based system for vision correction, comprising a UV laser source for providing laser radiation, a scanning system for lateral scanning of the laser radiation in the x- and y-directions, a focusing optical unit, a planning unit for generating planning data and a control unit designed to control the UVL-LVC system taking the planning data into consideration.

### BACKGROUND

UVL-LVC systems currently in use, for example the MEL systems by Carl Zeiss Meditec AG, the Amaris systems by Schwind eye-tech solutions GmbH or the Micron systems by Excelsius Medical GmbH, are systems for vision correction that have been successfully employed for a long time. Nevertheless, they have a number of deficiencies or disadvantages, for which solutions are intended to be revealed here.

A rigid laser beam guidance system is provided in current UVL-LVC systems, virtually without exceptions. Although this facilitates safe laser beam guidance, it requires that the patient on a patient couch is moved under a fixed system aperture in x, y, z coordinates by operation of said patient couch until the patient's eye intended to be treated is correctly positioned in relation to the optical axis of the system. An exception is formed by the system described in US 2013/0226157 A1, in which the laser arm, rigid per se, is positioned as a whole over the patient, albeit in a manner which still requires the positioning of the patient by way of the patient couch. However, for safety reasons, the latter frequently requires the patient couches to be electrically and/or mechanically connected to the laser base unit, which in turn requires a system certification and large amounts of space.

Additionally, there is a conventional manual static alignment of the eye in relation to cyclorotation in such UVL-LVC systems, i.e., without an automatic correction with the aid of registration data, by rotating the patient's head on the couch under visual monitoring. As a rule, this is not possible with great accuracy and a rotation of the head of the patient during the treatment is not precluded either, which is critical for the treatment results if there is no automatic cyclorotation correction.

Moreover, contact interfaces for affixing the eye are known in some current UVL-LVC systems. However, in such UVL-LVC systems, for example as described in US 2013/0226157 A1 and U.S. Pat. No. 9,592,156 B2, these are only implemented for eye stabilization purposes and do not adopt a truly active role. Work is carried out entirely without contact interfaces in many systems.

Large working distances between laser exit aperture and eye were realized with the introduction of spot-scanning UVL-LVC systems. This was also implemented against the background of using microkeratomes, which were used with the patient on the patient bed of the system, in order to cut the LASIK flap, that is to say an opening in the cornea that can be folded to one side. Among other things, this also required great working distances. Eye trackers for registering and compensating the eye movements were introduced at a later stage, and hence the eye movements during the ablation, which arose due to an unaffixed eye, were compensated. The overall optical system concept accompanying this—which in principle is very similar in the various systems—may also be considered to be disadvantageous.

Various problems arise in relation to eye tracking during the application thereof. Overall, the registration speed for the eye movement is limited and the adjustment of the scanner mirrors for correcting the pulse coordinate requires a finite amount of time. In the context of the system performance, the response to the eye movement (“response time”) is always delayed and hence never exact. In the case of current, fast eye tracker systems (with the repetition rate of approximately 1000 Hz), this is no problem for the lateral correction (x, y shift). However, the currently fastest system already responds to the limitations since the eye tracker speed is even below the limit for the repetition rate. In a manner of speaking, a prediction about the future movement of the eye is made on the basis of the preceding movement trajectories. Naturally, this is an oversimplification since eye saccades/nystagmus correspond to statistical movements of the eye in the broadest sense. This also highlights the fact that an increase in the repetition rate is hardly advantageous with current technology, even though this would be of interest for certain applications and in certain ablation timeframes (thermal controlled/mild ablation).

Moreover, purely lateral tracking is a limitation since the rotation of the eye about the z-axis (“dynamic cyclorotation”) and the roll movements about the horizontal and vertical eye axes have to be taken into account. Finally, the distance (z-distance) to the laser exit aperture may also vary, which can likewise be compensated for by appropriate tracking. Despite all technical intricacies and correction options, these systems however are not able to respond exactly to the eye position: The limited quality of the registration and the speed of the registration and correction do not allow this. Normally, the influence on the refractive results is very small and hardly detectable.

However, there are further problems with the eye tracking in the case of uncooperative patients, in the case of whom there are fixation instability, nervousness, cognitive deficits or problems with perceiving the fixation target. Then, the eye surgeons must affix the eyes during the ablation manually by application of a clamp or foam spatula in order to even make ablation possible. This occurs relatively frequently and is due to the fact that the eye movements are guided out of what is known as the (limited) “eye tracker hot zone” and the system must then stop.

In this context, the production of prismatic errors by the eye tracker must also be mentioned. The ablation profiles are not applied in the correct plane, that is to say not to the surface normal, i.e., perpendicular to the visual axis. This may happen if the patient by preference fixates in a largely fixed but “incorrect” direction, that is to say, e.g., permanently looks in a fixed direction that does not correspond to the center of the “fixation cloud” (depending on refractive deficit and treatment duration, the patient can no longer see the fixation target in focus during the operation).

Additionally, eye trackers do not work for all eyes on account of problematic eye color and/or lack of contrast. Percentage range outage rates of the eye tracker occur in practice. All that eye surgeons can then do is decide between terminating the operation and deactivating the eye tracker, which is then accompanied by the risk of an inaccurate correction.

Constant ambient conditions above the operation site cannot be set, or can only be set to a very qualified extent, in current systems.

It is known that the influence of varying ambient conditions, for instance air humidity and changes in the hydration state of the cornea accompanying this, the composition of the air (in the case of evaporation of solvents for example) or the temperature, on the refractive results may be significant. It is also known to be advantageous to ensure that the hydration state of the cornea is maintained during the ablation, or to ensure that drying out of said cornea is avoided. A distinction has to be made between two effects for the hydration: a) the physiological differences in the hydration of the cornea as a distribution over various patients, and b) the maintenance of the hydration during the ablation itself. Both influences lead to an increased variation in the refractive result (for example via an increased variation in the “attempted vs. achieved” prediction). The literature contains many investigations in this respect. Accordingly, the influence of the hydration of the cornea is significant in particular.

A further, very important factor that influences the refractive results is related to the amount and accumulation of ablation products (“debris”) over the ablated cornea, that is to say the operation site. It is sufficiently well known that the radiated-in UV ablation pulses are absorbed and scattered in the debris. This uncontrollably modifies the effective pulse fluence, which decisively controls the ablation process. This may lead to significant fluence deviations in the sequence of ablation pulses. By way of example, there is the risk of central steepening arising in the cornea post surgery in the case of myopia treatments (central islands). Therefore, current systems usually have aspiration structures or a combined air supply and aspiration structures for the debris. However, truly effective removal of the debris is only implementable to a limited extent in practice as a result of the large distance from the supply and removal lines. In principle, this would require a replacement of the entire air volume above the cornea to be treated between successive pulses in the case of pulse repetition rates between 500 Hz and 1000 Hz. As a worst-case scenario, this may lead to “skew” ablations and hence, for example, to induced coma or SIA (surgically induced astigmatism)—especially in the case of non-optimal or non-directed aspiration of the ablation products.

Moreover, there is the risk of corneal dehydration in the case of systems which additionally supply air. This can likewise only be partly prevented. Overall, the operation site cannot be decoupled from the remaining operative surroundings (e.g., airflows in the room) as a result of the open arrangement of current systems either.

The sterile and secure placement of the flap is of extraordinary importance for a LASIK procedure. Typically, a flap is only 100 μm thick and, following the LASIK incision, only fastened to the cornea by way of a very narrow “hinge”. Maintaining the hydration of the flap is very important for pathological reasons and also for maintaining the shape of the flap since dehydrated flaps shrink within seconds. Following the treatment, a shrunken flap no longer “fits” well into the stromal bed (which naturally is also due to the change in shape of the stromal surface as a result of the ablation), which may in turn lead to postsurgical complications (e.g., epithelial ingrowth). Where possible, flaps should not be bent, pulled or otherwise stressed either. Hence, experts these days hardly still use the “calzone technique” that was employed in the past. Moreover, it is imperative to avoid the flap coming to rest in possibly non-sterile regions of the eye. Despite the sterile preparation of the eye, this may nevertheless occur, for example as a result of the tear film or contact with non-sterile parts of the lids.

In want of a solution that is integrated in the current systems, some users cut their own flap repositories from sterile foam spatulas (or similar materials), which are then moistened and serve as safe and sterile rest for the sensitive flap. Thus, a solution is sought after in order to put an end to this situation.

On account of the relatively large working distances A of existing UVL-LVC systems from the eye, there is hardly a difference there in the focusing plane. Therefore, these UVL-LVC systems can be considered to be virtually telecentric on the image side.

On account of the relatively large working distance of known UVL-LVC systems from the patient's eye, it is difficult to impossible to use reflections from the cornea of the patient's eye for analysis purposes, which as a consequence makes a centration difficult as well. The influence of an inaccurate centration is known and discussed multiple times in the literature. The “common” argument that centration errors, that is to say deviations of the ablation center from the target positions on the cornea, as are typically given by the “ophthalmic pole” for centration on the visual axis and as are referred to as decentrations below, have no influence on spherical corrections is physically true only in certain cases, for example spherical corrections on spherical corneas. However, the visual physiology, inter alia, is not considered in this case. As a rule, decentrations will lead to a shift in the physiological visual axis. When processing the visual impression in the brain, the eye is “rotated” by the eye muscles such that the light from the fixated object continues to fall on the point of sharpest vision, which in principle compensates the prismatic offset (“tip/tilt”). This may lead to problems in the case of binocular vision (stereopsis), for example, which problems are known from investigations in relation to poorly centered spectacle lenses, for example.

At the latest in the case of aspherical corrections on ellipsotoric corneas, which corresponds to the real, actual scenario, a decentration also leads purely physically to a non-attainment of the sought-after correction.

There is also no need to explain further that decentrations have a significant influence on the results of “customized ablation”, as this leads to the induction of higher aberrations (“night vision complaints”, etc.) and hence also to an influence on the refractive result. Hence, a centration that is as exact as possible is the absolute basic precondition for a good result in the case of both topography and wavefront corrections.

Aberrations (or optical modes) couple under decentration. As a result of defocus and cylinder coupling to higher-order aberrations (coma, spherical aberration, higher-order astigmatisms) or of aberrations occurring in natural (aspherical) eyes, decentrations in real eyes are critical, even in the case of pure spherocylindrical corrections. By way of example, in the case of a decentration, coma couples to astigmatism and defocus or spherical aberration couples to coma, astigmatism and defocus. Here, a few examples should be provided for an optical diameter of 6 mm, the examples initially only demonstrating the effects for aberrations up to the 3rd order (the sum of the mode indices is 4). The calculations follow from the coordinate transformation of optical modes:
A shift of 0.25 μm coma Z(3,1) by 0.3 mm (horizontal/vertical) leads to a defocus of approximately −⅛ dpt.
A shift of 0.25 μm coma Z(3,1) by 0.3 mm (horizontal/vertical) leads to a cardinal astigmatism (Z(2,2)/Z(2,−2)) of ⅛ dpt.
A shift of 0.5 μm coma Z(3,1) by 0.5 mm leads to a defocus of approximately −0.3 dpt.
A shift of 0.4 μm coma Z(3,1) by 0.5 mm (horizontal/vertical) leads to a cardinal astigmatism (Z(2,2)/Z(2,−2)) of 0.3 dpt.
A shift of 0.6 μm spherical aberration Z(4,0) by 0.4 mm leads to a defocus of approximately −⅛ dpt.
A shift of 0.6 μm spherical aberration Z(4,0) by 0.4 mm (horizontal/vertical) leads to a cardinal astigmatism (Z(2,2)/Z(2,−2)) of approximately ⅛ dpt.

Until now, only optical modes manifesting themselves in ablation profiles in the optical zone were considered. The transition zones have not yet been mentioned. However, in this context decentrations also mean that transition zones may reach into the optically active zone, especially in the case of hyperopia corrections. This then leads to disturbances in the case of mesopic to scotopic light conditions (“night vision complaints post surgery”; this does not refer to night myopia), and hence to patient dissatisfaction.

Pupil centrations (centration in relation to the CSC, “corneal sighting center”) can be brought about well and reliably in refractive surgery by application of eye tracking systems (“eye tracker”) as integrated pupil recognition. However, this type of centration is not the preferred choice since it has in the meantime been settled in the art that a centration in relation to the ophthalmic pole (visual axis, coaxially sighted corneal light reflex, “CSCLR” condition, see below) would be correct. Experience has shown that small and medium myopia corrections are very uncritical in this case. Relatively large astigmatisms and myopia corrections and, in particular, hyperopia corrections are more difficult. This is because hyperopic eyes are typically characterized by a non-negligible angle between the pupil axis (also referred to as pupillary axis) and the visual axis (“angle kappa”). In this case, corneal sighting center and ophthalmic pole are no longer sufficiently close together, leading to a difference between “angle lambda” and “angle kappa”. Moreover, the pupil and the pupil center are no fixed marking. Both vary with the lighting conditions.

A centration on the visual axis is implemented in the case of the current laser systems by searching for the first Purkinje image of the fixation laser (“target”). Under patient fixation, the recommended CSCLR condition is met when the Purkinje image comes into the center of the system optical unit and the optical system axis and the visual axis become coaxial. Finding this reflection is not trivial in current systems. This is made even more difficult in the case of less cooperative patients, for example who exhibit weak fixation, since the reflection then continually disappears. Moreover, the “parallax error” of the surgical microscope, that is to say different directions for the reflection in the right and left observer eye as a result of the binocular arrangement, makes a correct alignment more difficult.

There is no automatic centration in accordance with CSCLR by way of Purkinje images, and also no prospect thereof, in current systems. As it is carried out reliably and automatically by the systems, in contrast to CSCLR centration by use of Purkinje images, this is probably one of the reasons why the pupil centration—despite the problems accompanying this—is currently preferred by many users.

A manual centration on the vertex (CV), which generally represents a reference center for the topography, by entering displacement coordinates is frequently carried out for topography-guided corrections, but also in the case of standard spherocylindrical corrections. The latter is possible since, in the case of normal eyes, the positions of the CV (“corneal vertex”, that is to say the intersection point of the keratometric axis on the cornea under patient fixation) and the ophthalmic pole (OP), that is to say the visual axis, are sufficiently close together. This in turn is due to the fact that the center of curvature of the cornea approximately coincides with the second image-side optical node of the eye (cf. Gullstrand, Liou-Brennan eye models). An automated centration with respect to the vertex is not found currently. The user frequently shifts the treatment center manually purely on the basis of the visual comparison with a topography measurement. Or they enter displacement coordinates into the system, which are generally specified in relation to the pupil center (CSC) and, for example, are taken from a topography measurement. A problem in both cases is that the pupil diameter during the topography measurement does not correspond to the pupil diameter under the laser on account of differences in lighting. The frequently arising shift of the pupil center with the pupil size then leads to a non-optimal centration as the corneal vertex has not been determined correctly.

Current UVL-LVC systems offer no method of tomographic alignment of the anterior chamber and the tomographic centration. By way of example, this may be important in the case of corneal irregularities (e.g., caused by traumas or brief swelling of the corneal surface as a result of bubbles following femtosecond laser flap generation), which lead to the corneal vertex and/or the Purkinje image not being found correctly or, expressed differently, a position being identified as the vertex which does not correspond to the normal physiological vertex position. The pure “surface information” from the cornea is then not suitable, or not sufficiently well suitable, for a determination of the optimal centration.

### SUMMARY OF THE INVENTION

Example embodiments of the present invention describe apparatuses which address the aforementioned problems of currently used UVL-LVC systems. In particular, embodiments of the present invention facilitate improvement of the predictability of the refractive results following a correction by application of UVL-LVC systems.

A first aspect of the invention relates to a first variant of a focusing optical unit for a UV laser-based system for vision correction (UVL-LVC system) having a UV laser source for providing laser radiation (for example pulsed laser radiation) and a scanning system for lateral scanning of the laser radiation in the x- and y-directions. The scanning system can be additionally designed to scan in the z-direction. The focusing optical unit serves to focus the laser radiation in a focal field. A laser beam provided by the laser source and shaped by the focusing optical unit has what is known as a “spot” in the focal field (which is also referred to as focus plane, focal plane or focusing plane). For example, the spot has a well-defined “spot diameter” or a “spot size” of 0.3 mm to 1.5 mm, for example 0.5 mm to 1.0 mm. Thus, the focal field is described by the requirements in respect of a lateral extent of the laser radiation. The numerical aperture of a laser beam provided by the focusing optical unit can be less than 0.1, for example less than 0.08. This yields a great depth of field for the laser beam. Hence, the focus of a laser beam need not be in the focal field; it may also be situated a few depths of field in front of or behind the focal field. The lateral extent of the spots is also maintained in that case.

The focal field can be partly or completely identical to a work surface suitable for a treatment for vision correction. For therapy, the eye to be treated or the cornea thereof is typically positioned in or close to this work surface.

According to the invention, the focusing optical unit has a first lens arrangement, which is designed to provide a convergent focal field. The convergence of the focal field is characterized in that a totality of laser rays (for different locations in the focal field, provided by way of the scanning system of the UVL-LVC system) leaving the focusing optical unit run convergently to one another. This means that the totality of the laser rays (and their beams) leaving the focusing optical unit have a common point of intersection. There is no such point of intersection for a telecentric or divergent focal field.

The first lens arrangement of the focusing optical unit may have one or more lenses. The first lens arrangement is for example designed to guide UV light without noticeable losses. To this end, the transmission of the first lens arrangement is particularly high, especially in a wavelength range between approximately 193 nm and 213 nm. By way of example, the first lens arrangement may contain glasses with CaF or fused silica and may be provided with suitable optical coatings.

The focusing optical unit according to the invention follows an entirely novel approach for the ablation geometry. An optical concept differing from all other UVL-LVC systems is implemented in this case. This novel concept allows fundamental improvements to be made in many areas of UVL LVC; in particular, an improvement in the predictability of the refractive results is made possible.

For elucidation purposes, the characteristics of the ablation geometry allowed by the focusing optical unit according to the invention are described in comparison with optical systems for UVL-LVC systems according to the prior art. In the case of conventional UVL-LVC systems, the rays are incident on the cornea of the eye at a significant angle (in relation to a perpendicular incidence) since the typical radius of curvature R_{C} of the human eye is approximately 7.86 mm. The working distance Δ of typically 250 mm is also relatively large for these systems.

In the prior art, the focusing optical unit is typically configured such that there is telecentric focusing of the ablation pulses. Expressed differently: if an appropriate beam (also referred to as laser beam) is assigned to each xy-position on the eye provided by the scanner, these beams are only displaced and not tilted with respect to one another. Telecentric focusing occurs. The centroid rays (or chief rays) of the beams have angles with respect to the surface normal of the cornea of the eye which become ever larger with increasing distance from the apex of the cornea and which deviate ever more strongly from a perpendicular incidence on the cornea.

Alternatively, the focusing optical unit in the prior art is designed such that there is divergent focusing of the ablation pulses. Expressed differently, if the totality of the beams are considered, these leave the focusing optical unit in divergent fashion. Hence, the angles between the centroid rays of the beams and the surface normals of the cornea of the eye become even larger with respect to a perpendicular incidence with increasing distance from the apex of the cornea than in the case of the above-described focusing optical unit (with telecentric focusing) according to the prior art.

This is very different in the case of the focusing optical unit according to the invention. As stated previously, the latter has a convergent focal field. As a result of the convergence of the focal field, the laser radiation is incident on the cornea at an angle that is closer to perpendicular incidence than what can be achieved according to the prior art, for all locations in the focal field. The advantages of this geometry are discussed below.

For example, the first lens arrangement of the first variant of the focusing optical unit according to the invention has more than one lens for the provision of the convergent focal field. For this purpose, the lenses can be configured like in the case of a microscope objective which provides the convergent focal field on the image side (on the side of the eye or its cornea) in the case of an oblique incidence of the laser ray (into the focusing optical unit) on the object side. The object-side oblique incidence into the focusing optical unit can be realized by way of suitable scanners of the UVL-LVC system. Thus, laser beams deflected by the scanners are directed to the convergent focal field through the optical units as “spots”.

The challenge lies in reducing the physical aberrations in the case of oblique incidence and the aperture errors of the focusing optical unit because the imaging quality of the optical unit otherwise quickly reduces significantly outside of the paraxial range, that is to say with increasing distance from an optical axis. By way of example, this would lead to the spots produced by the system in the convergent focal field varying both in terms of size and shape (and hence in terms of the fluence distribution), for example as a result of astigmatic aberrations. Moreover, the spot positions would be displaced by stigmatic distortions (e.g., a barrel distortion, pincushion distortion). In the combination of the effects, the (sought-after) spots with the corresponding unchanging quality would not be achieved depending on the scanning direction in the scan field.

To reduce the aberrations to the necessary degree (requirements in respect of the diameter changes and form changes of the spots in the convergent focal field), that is to say in order to achieve a sufficient quality of the imaging, the focusing optical unit can either be composed of spherical optical units and/or be realized by aspherical optical units or contain aspheres. Using the latter, it is possible to reduce the installation size of the objective (focusing optical unit) in particular. In the case of a desired spot diameter of typically round shape with a full width at half maximum (FWHM) ranging from 0.3 mm to 0.8 mm in the focal field (in the case of a supergaussian fluence distribution in the laser beam), the diameters and shape changes should be located in the range below 20%, for example below 10% (RMS radius deviations <50 μm). The optical units may additionally or alternatively have free-form surfaces. Alternatively, the focusing optical unit may also have (imaging) diffractive elements, for example in the form of radially symmetric diffractive structures applied to a curved lens.

According to an example configuration of the first variant of the focusing optical unit, the convergent focal field has a diameter of at least 6 mm, for example at least 8 mm, in another example at least 10 mm.

In this way, a diameter of approximately 6 mm (or 8 mm, 10 mm) can be obtained in the work surface suitable for a treatment for vision correction.

In this case, the focusing optical unit is for example configured such that the above-described quality of the imaging is observed over the entire diameter of the curved surface.

For example, the optical system of the UVL-LVC system is designed such that the laser radiation is fed to the focusing optical unit according to the invention such that the claimed diameter of the convergent focal field is served. The scanning system can be embodied accordingly to this end.

The optical system of the focusing optical unit with such a diameter of the convergent focal field allows a fixed offset to be applied to the scanner coordinates in order thereby to shift the treatment center with respect to an optical system axis of the UVL-LVC system, which for example runs centrally through the focusing optical unit.

According to an example configuration of the first variant of the focusing optical unit, each location in the convergent focal field has a local center of curvature on the side of the convergent focal field facing away from the focusing optical unit. In other words: If the convergent focal field is locally assigned a curvature—at the location where the laser radiation impinges on the convergent focal field—then this local curvature can be assigned a local center of curvature (for example, by way of an approximation to a spherical curvature, with the center of curvature corresponding to the center of the sphere or ball). As considered from the first variant of the focusing optical unit, this center of curvature is behind the convergent focal field. The curvature of the convergent focal field consequently has the same sign as the curvature of the eye whose vision should be corrected. The convergent focal field has a finite radius of a focal field curvature. For example, the convergent focal field has a focal field curvature with a radius R_{S} ranging from 8 mm to 50 mm, for example from 10 mm to 30 mm, in another example from 12 mm to 20 mm. The focal field curvature may also have a radius R_{S} from 6 mm to 25 mm, from 7 mm to 20 mm, or from 8 mm to 16 mm. The focal field curvature may vary locally over the focal field within the specified limits. For example, the focal field has a focal field curvature that corresponds to a sphere or an asphere; an aspherical form is preferred, in particular, when a large focal field diameter (for example of at least 6 mm) is provided.

In a a particular example configuration, the focal field curvature for the ablation is designed such that the latter corresponds to the typical radius of curvature of the cornea R_{C} (“convergent focal field ablation”). This realizes a virtually perpendicular incidence of the incident laser radiation on the cornea. However, on account of limitations when parameterizing the optical system of the focusing optical unit and the required working distance Δ for clinical practice, at least focal field curvatures R_{S} that are very close to R_{C} are possible. This is desirable in order to obtain a significant reduction in the fluence losses (see below), in order to achieve the advantages described there.

If the focal field has a curvature R_{S}, it is possible to determine a difference radius of curvature R_{Δ} with respect to the cornea with the radius of curvature R_{C}. Said difference curvature corresponds to an “effective” corneal curvature for light incident from the z-direction (i.e., parallel to an optical axis of the focusing optical unit). As a result of the difference calculation, the cornea is “bent” upward—figuratively speaking—by the focal field radius of curvature R_{S}, then allowing the calculation of a fluence loss to be carried out in a simplified manner, that is to say on the basis of an “effective” corneal curvature (with an effective corneal radius of curvature R_{Δ}). This effective corneal curvature is less than the actual corneal curvature. Consequently, with a typical corneal radius of curvature of R_{C}=7.86 mm, values of approximately R6hd Δ≈450 mm to approximately R_{Δ}≈15 mm arise for the effective corneal curvature for focal field radii of curvature with values between approximately 8 mm and approximately 16 mm. The advantage of an improved fluence loss function appears clearly in these ranges, as is yet to be shown below.

The invention relates to a second variant of a focusing optical unit for a UV laser-based system for vision correction (UVL-LVC system) having a UV laser source for providing laser radiation (for example pulsed laser radiation) and a scanning system for lateral scanning of the laser radiation in the x- and y-directions. The scanning system can be additionally designed to scan in the z-direction. The second variant of the focusing optical unit serves to align the laser radiation. According to the invention, the second variant of the focusing optical unit has a second lens arrangement, which is designed to provide perpendicular incidence of laser radiation on a curved surface. This means that a chief ray (or centroid ray) of a beam of laser radiation is directed to the curved surface by the second variant of the focusing optical unit, in such a way that the angle between the chief ray and the surface normal at the location where the laser radiation is incident on the curved surface forms an angle of no more than 10°, for example no more than 5°, in another example no more than 2°. According to the invention, each location on the curved surface has in this case a local center of curvature on the side of the curved surface facing away from the second variant of the focusing optical unit. In other words: If the curved surface is locally assigned a curvature—at the location where the laser radiation impinges on the curved surface—then this local curvature can be assigned a local center of curvature (for example, by way of an approximation to a spherical curvature, with the center of curvature corresponding to the center of the sphere or ball). As considered from the second variant of the focusing optical unit, this center of curvature is behind the curved surface. The curvature of the curved surface consequently has the same sign as the curvature of the eye whose vision should be corrected. The curved surface has a finite radius of a surface curvature.

For example, the curved surface has a surface curvature corresponding to that of a sphere or an asphere. An aspherical form is preferred, in particular, if a large diameter of the curved surface (e.g., at least 6 mm) is provided. The local centers of curvature can also be located along the continuation of the respective chief ray.

Attention is drawn to the fact that the second variant of the focusing optical unit need not necessarily provide a focus of the laser radiation on the curved surface. Rather, all rays of a beam may also be incident on the curved surface in perpendicular fashion. When using a low beam quality UV laser source, for example an excimer laser, the beams may also have a focus on the curved surface. By way of example, a solid-state laser can also be used as a UV laser source. At the location of incidence on the curved surface, the laser radiation advantageously has a diameter of 0.3 mm to 1.5 mm, for example of 0.5 mm to 1.0 mm. The location of impingement is often referred to as “spot” and its diameter is referred to as “spot diameter” or “spot size”. The spot size may serve as a measure for the size with which the curvature on the curved surface is averaged for the purposes of determining the local curvature or the center of curvature.

The curved surface can be partly or completely identical to a work surface suitable for a treatment for vision correction. For therapy, the eye to be treated or the cornea thereof is typically positioned in or close to this work surface.

The second lens arrangement of the second variant of the focusing optical unit may have one or more lenses. The second lens arrangement is for example designed to guide UV light without noticeable losses. To this end, the transmission of the lens arrangement is particularly high, especially in a wavelength range between approximately 193 nm and 213 nm. By way of example, the lens arrangement may contain glasses made of CaF or fused silica and may be provided with suitable optical coatings.

As already discussed above, the chief rays in focusing optical units according to the prior art have angles of incidence deviating ever more from perpendicular incidence with increasing distance from the apex of the cornea because the point of intersection of the chief rays is located in the optical unit. This is very different in the case of the second variant of the focusing optical unit according to the invention. In this case, the curved surface (and the sign of the curvature) leads to the deviation from perpendicular incidence of laser radiation on the cornea of the eye being significantly reduced in relation to the prior art because the point of intersection of the chief rays (for increasing distance from the apex of the cornea) is located behind the corneal apex.

For example, the second lens arrangement of the second variant of the focusing optical unit according to the invention has more than one lens for the provision of the curved surface. To this end, the lenses can be configured in such a way that there is perpendicular impingement of the curved surface on the image side (on the side of the eye or its cornea) in the case of an oblique incidence of the laser ray (into the second variant of the focusing optical unit) on the object side. The object-side oblique incidence into the second variant of the focusing optical unit can be realized by way of suitable scanners of the UVL-LVC system. Thus, laser beams deflected by the scanners are directed to the curved surface through the optical units as “spots”.

Requirements and solutions in respect of the imaging quality for the second variant of the focusing optical unit correspond, in principle, to the requirements and solutions for the first variant of the focusing optical unit (and its configurations).

For example, the second variant of the focusing optical unit also has the features of the first variant of the focusing optical unit (and its configurations). In this case, parts of the first lens arrangement can be identical with parts of the second lens arrangement. The first and second lens arrangement can also be completely identical. The convergent focal field and the curved surface can be partly or completely identical.

According to an example configuration of the second variant of the focusing optical unit, the curved surface has a diameter of at least 6 mm, for example at least 8 mm, in another example at least 10 mm.

In this way, a diameter of approximately 6 mm (or 8 mm, 10 mm) can be obtained in the work surface suitable for a treatment for vision correction.

In this case, the second variant of the focusing optical unit is for example configured such that the above-described quality of the imaging is observed over the entire diameter of the curved surface.

In another example, the optical system of the UVL-LVC system is designed such that the laser radiation is fed to the second variant of the focusing optical unit according to the invention such that the claimed diameter of the curved surface is served. The scanning system can be embodied accordingly to this end.

The optical system of the second variant of the focusing optical unit with such a diameter of the curved surface allows a fixed offset to be applied to the scanner coordinates in order thereby to shift the treatment center with respect to an optical system axis of the UVL-LVC system, which for example runs centrally through the focusing optical unit.

According to an example configuration of the second variant of the focusing optical unit, the curved surface has a surface curvature with a radius R_{F} ranging from 8 mm to 50 mm, for example from 10 mm to 30 mm, in another example from 12 mm to 20 mm. The surface curvature may also have a radius R_{F} from 6 mm to 25 mm, from 7 mm to 20 mm, or from 8 mm to 16 mm. The surface curvature may vary locally over the curved surface within the specified limits.

In a particular example configuration, the surface curvature for the ablation is designed such that the latter corresponds to the typical radius of curvature of the cornea R_{C} (“convergent focal field ablation”). This realizes a perpendicular incidence of the incident laser radiation on the cornea. However, on account of limitations when parameterizing the optical system of the second variant of the focusing optical unit and the required working distance Δ for clinical practice, at least surface curvatures R_{F} that are very close to R_{C} are possible. This is desirable in order to obtain a significant reduction in the fluence losses (see below), in order to achieve the advantages described there.

The observations in relation to a difference radius of curvature R_{Δ} for a first variant of the focusing optical unit with a focal field curvature R_{S} also apply to the second variant of the focusing optical unit, described here, with a surface curvature R_{F}.

In an example configuration, the first or second variant of the focusing optical unit has a working distance Δ ranging from 20 mm to 55 mm, for example from 20 mm to 50 mm. Additionally or as an alternative, the focusing optical unit has an optical aperture of greater than 40 mm, for example greater than 50 mm, in another example greater than or equal to 60 mm.

Focusing optical units according to the prior art have significantly longer working distances and typically have smaller apertures of the focusing optical unit. This also leads to the optical acceptance angle for the return of corneal reflections into the optical system being very small. By contrast, the focusing optical unit according to the invention (in both variants) allows particularly good return of reflections (e.g., the Purkinje image) into the remaining UVL-LVC system through the focusing optical unit. This allows a significantly improved alignment of the UVL-LVC system (or of the focusing optical unit) with respect to the eye of the patient and consequently makes possible a further improvement in the predictability of the refractive results following a correction. In a particular example, the convergent focal field (in a configuration of the first variant of the focusing optical unit) additionally has a focal field curvature with a radius R_{S} ranging from 8 mm to 50 mm, for example from 10 mm to 30 mm, in another example from 12 mm to 20 mm. For the application, these values for the focal field radius of curvature R_{S} yield further improved conditions for the detection of a ray reflected by the cornea.

In a particular example, the curved surface (in a configuration of the second variant of the focusing optical unit) additionally has a surface curvature with a radius R_{F} ranging from 8 mm to 50 mm, for example from 10 mm to 30 mm, in another example from 12 mm to 20 mm. For the application, these values for the focal field radius of curvature R_{F} yield further improved conditions for the detection of a ray reflected by the cornea.

The UV laser of the UVL-LVC system may have a spectral width of approximately 0.5 nm, for example. This may lead to the occurrence of transverse chromatic aberrations. Advantageously, the transverse chromatic aberrations can be compensated by way of a combination of lenses of the focusing optical unit, with the lens materials of the lenses having both different refractive indices and different Abbe numbers in the spectral range of the UV laser. Therefore, in an advantageous configuration of the focusing optical unit, the latter comprises a first lens and a second lens, wherein the first lens has a first lens material with a first refractive index and a first Abbe number and the second lens has a second lens material with a second refractive index and a second Abbe number, with the first refractive index differing from the second refractive index and the first Abbe number differing from the second Abbe number. Here, the different refractive indices must be present in this case in the range of the wavelength of the UV laser. CaF and fused silica materials are mentioned here in exemplary fashion.

In a particular example development of the focusing optical unit, the first lens has a negative optical power, the second lens has a positive optical power and the first refractive index is greater than the second refractive index. In this case, too, the refractive indices are considered in the range of the wavelength of the UV laser. The transverse chromatic aberration of the focusing optical unit can be corrected particularly well using the combination according to the invention of the optical powers and refractive indices.

According to a configuration of the focusing optical unit (in both variants), the first or second lens arrangement is designed to guide visible light without noticeable losses, also while avoiding a degradation of the optical components. To this end, lenses of the focusing optical unit may for example have a coating that provides high transmission (for example greater than 80%, in another example at least 90%) for both the spectral range of the UV laser and for the used visible light.

According to a configuration, the first or second variant of the focusing optical unit has at least two lens groups along a beam path with a non-imaging optical element being arranged therebetween. A lens group can comprise one or more lenses. The non-imaging optical element can be a plane mirror, a beam splitter or an optical filter (for example for polarization or wavelengths) or a retarder (for example a quarter-wave plate or a half-wave plate), which is embodied as a plane parallel plate (or mirror). This allows, for example, the realization of a compact structure of the focusing optical unit or the guidance of further light (for example visible light for fixation light) over parts of the same optical unit or the filtering of the guided light (e.g., of the laser source) or the influencing of the polarization of said guided light.

What follows is a brief explanation as to how fluence losses are reduced or eliminated by the focusing optical unit according to the invention; the explanations apply to both variants of the focusing optical unit. A pulse ablation shape corresponds to an ablation-effective fluence distribution of the radiated-in ablation laser pulse in a plane perpendicular to the direction of incidence. As a result of the geometry of the irradiation of the cornea, the pulse ablation shape deforms into a projected pulse ablation shape (also referred to as “pulse ablation footprint on cornea”). Hence, the fluence distribution on the cornea changes with respect to the “pulse ablation shape” that was radiated in. By way of example, this can be calculated from a given pulse shape with the aid of the blow-off model. For an infinitesimal surface element dA in the pulse ablation shape, the geometry loss is modeled as a cosine function and the following applies: cos(α)=dA/dA′, with the angle of incidence α (angle of incidence with respect to the local surface normal on the cornea) and an infinitesimal surface element dA′ of the projected pulse ablation shape. For a focusing optical unit with a convergent focal field (or perpendicular impingement of a curved surface) according to the invention, only small angles of incidence α arise in comparison with the prior art, and so the “pulse ablation footprint on cornea” only deviates little from the radiated-in pulse ablation shape.

Hence, the focusing optical unit according to the invention (in all variants and configurations) has lower geometry losses than solutions according to the prior art.

Fresnel losses, which can be calculated with the aid of the Fresnel equations with knowledge of the refractive indices of the air and cornea (or stroma) and the angles of incidence, may also occur in addition to geometric losses. The polarization of the light also needs to be taken into account. For polarized light, simulations have shown (as discussed below) that the losses due to the focusing optical unit according to the invention can be reduced and that, in particular, a dependence on the pupil radius is reduced.

In summary, it is possible to show that the focusing optical unit according to the invention (in all variants and configurations) produces fewer geometry losses than solutions according to the prior art. Furthermore, the Fresnel losses are also significantly lower. In particular, there is a significantly lower dependence on the pupil radius for both effects. The focusing optical unit according to the invention therefore advantageously allows an improvement in the predictability of the refractive results following a correction by application of UVL-LVC systems.

A second aspect of the invention relates to a planning unit for generating planning data for a UV laser-based system for vision correction (UVL-LVC system). In this case, the UVL-LVC system has a UV laser source for providing laser radiation and a scanning system for lateral scanning of the laser radiation in the x- and y-directions. The scanning system can be additionally designed to scan in the z-direction. Furthermore, the UVL-LVC system has a focusing optical unit for directing the laser radiation to a work surface. In this case, the work surface is a surface in space suitable for a treatment for vision correction. For therapy, the eye to be treated or the cornea thereof is typically positioned in or close to this work surface. The work surface may be curved. Moreover, the UVL-LVC system has a control unit that is designed to control the UVL-LVC system taking the planning data into consideration. To this end, the control unit may provide control signals for the UV laser source and the scanning system via control lines. Consequently, a laser focus can be displaced in the work surface on the basis of the planning data. Furthermore, a UV laser source power (by way of example, by changing the pump power in the case of a pumped laser source or with the aid of a variable attenuator) or else, optionally, a pulse rate can be controlled on the basis of the planning data. The control unit may be a computer comprising a processor and a memory.

According to the invention, the planning unit is designed to consider geometry losses and/or Fresnel losses when calculating the planning data. In this case, the planning unit can be designed to calculate the geometry and Fresnel losses on the basis of data from the focusing optical unit, the work surface and a curvature, determined preoperatively, of the structure in the eye to be treated, for example the cornea or the stroma.

Additionally or as an alternative, a spatial extent of the laser radiation in the work surface is taken into account when calculating the planning data.

In this case, it is possible to consider a variation of the laser radiation within the laser spot and/or diameter changes and shape changes of the spots in the work surface. By way of example, such changes may occur when the laser power changes and/or depending on the position of the laser spot in the work surface.

According to the invention, the aforementioned effects are taken into account in such a way that their influence on fluence losses is compensated (by way of an energy correction). By way of example, the power of the laser radiation can be altered locally to this end. Additionally or as an alternative, it is possible to adjust a dwell time of the laser radiation at a location in the work surface (by way of control signals to the scanning unit); in this way, it is possible, for example, to adjust the spatial distance between laser pulses or the number of laser pulses at the same location. Consequently, the planning unit can advantageously realize a fluence loss compensation.

Furthermore, the planning unit has an interface, by application of which the planning data can be made available to the control unit.

The planning unit may have a computing unit for calculating the planning data.

In this case, the planning unit according to the invention is not restricted to a UVL-LVC system with a focusing optical unit according to one of the above-described variants and configurations according to the invention. Rather, the planning unit also reveals—as shown below—an advantageous effect for systems having a focusing optical unit according to the prior art.

The planning unit can be part of the control unit of the UVL-LVC system. It may also have a separate computer with processor and memory.

Attention is drawn to the fact that the planning data can be created by the planning unit independently of the actual laser-surgical intervention on a patient's eye.

As will still be discussed in detail below, simulations have shown that it was possible with the aid of the planning unit according to the invention to significantly reduce, in relation to the prior art, the deviation of an effective etch rate from a normalized target etch rate as a function of the (preoperative) radius of curvature of the cornea for a UVL-LVC system with a fluence loss compensation according to the invention. This effect is improved even further if use is made of a focusing optical unit according to the invention.

According to an advantageous configuration of the planning unit, a decentration of an optical axis of the eye with respect to the visual axis or irregularities in the cornea and/or biomechanical effects of the eye are also considered when calculating the planning data. Furthermore, the planning unit can also calculate correction data required in the case of a decentration of the eye with respect to the optical axis of the focusing optical unit. These can then be used, for example, if an offset of the patient's eye occurs.

In a further configuration, the planning unit has an interface, by operation of which information data can be provided to the planning unit from the control unit of the UVL-LVC system. By way of example, the information data can be information about an offset in the case of a non-coaxial alignment of the patient's eye (or fixation of the patient's eye) with respect to the focusing optical unit (corresponding to the CSCLR condition). Additionally or as an alternative, this can relate to image data or information about a reflection; the planning unit can be designed to calculate the offset. According to a configuration of the planning unit, the latter is designed to consider the offset when calculating the planning data.

According to an example configuration of the planning unit, the UVL-LVC system has a focusing optical unit according to any one of the above-described variants and configurations. The ablation geometry with the convergent focal field and/or curved surface together with the optimized fluence loss compensation function according to the invention (realized by way of the planning unit according to the invention) enables a virtually perfect realization of the target ablation rate. It is possible to reduce the variation in the prediction of refractive results (reduced variation in “attempted vs. achieved”). As shown below, a reduction in the variation of the “achieved outcome” versus “attempted” may be of the order of up to ±0.25 diopters in individual cases—purely as a result of an improved energy correction on the basis of the ablation geometry according to the invention!

The almost perfect ablation geometry results in practical elimination of the influence of the optical system on nomograms (“rectification of nomograms”) since a deviation of the refractive results no longer occurs in this respect and consequently is no longer reflected in the nomograms. Ultimately, the “rectification” of the nomograms moreover leads to an improved determinability, acquirability and correctability of other relevant influencing variables of the refractive correction by way of nomograms (reduction in the “bias” in the nomograms) and to a reduction of disadvantageous interactions between various nomogram influencing variables.

A third aspect of the invention relates to a UV laser-based system for vision correction (UVL-LVC system). The UVL-LVC system comprises a UV laser source for providing laser radiation and a scanning system for lateral scanning of the laser radiation in the x- and y-directions. The scanning system can be additionally designed to scan in the z-direction. Furthermore, the UVL-LVC system has a focusing optical unit according to any one of the above-described variants and configurations. The UVL-LVC system moreover comprises a planning unit for generating planning data according to any one of the above-described configurations. Furthermore, the UVL-LVC system has a control unit that is designed to control the UVL-LVC system taking the planning data into consideration.

For example, the optical unit of the UVL-LVC system is designed such that the focusing optical unit, in conjunction with a remaining system optical unit, is able to effectively “collect”, by way of the system, the light reflected by the cornea (e.g., Purkinje images) and when necessary guide said light back to a base unit of the optical system by virtue of the same optical unit as for the laser beam delivery to the eye. Inter alia, this is made possible by virtue of the fact that the scanners, unlike in solutions according to the prior art, are able to be integrated at the start of the beam path (from the view of the UV laser source).

According to a configuration of the focusing optical unit, the latter is configured so that an observation of the operation site in the visible spectral range is possible therethrough. To this end, there is suitable imaging on a camera. Additionally or as an alternative, the UVL-LVC system has a surgical microscope for visual observation. In this case, the focusing optical unit acts as a common “front lens” of the surgical microscope. In this case, it is also possible to make use of auxiliary lenses which improve the imaging of the focusing optical unit for the purposes of observation in the visible spectral range to the extent that is necessary in order to obtain a sufficiently high resolution. However, instead of the visible spectral range, it is also possible to use, for example, IR light and cameras for the observation of the operation site.

In a further configuration of the focusing optical unit and the remaining system optical unit, the optical unit is configured such that both visible light and the UV laser radiation between approximately 193 nm and 213 nm can be guided without noticeable losses, also while avoiding a degradation of the optical components. To this end, the optical units can for example include CaF (calcium fluoride) or fused silica and may be provided with suitable optical coatings.

According to a configuration, the UVL-LVC system may have a base unit which comprises the UV laser source and one or more articulated arms, by operation of which the laser ray is guided to the focusing optical unit. The articulated arms may be interconnected by way of rotary joints. Then, the planning unit is particularly advantageously designed to consider a decentration of the laser ray in a zero position of the scanners, produced by the position of the articulated arms, with respect to an optical axis of the focusing optical unit. By way of example, the articulated arms may have sensors to this end, the latter detecting the position of the articulated arms with respect to one another and with respect to the base unit and making available the corresponding measurement values to the planning unit (optionally via the control unit).

According to a further configuration, the UVL-LVC system has a contact interface. The contact interface permits an alignment and/or a fixation of the eye with respect to the UVL-LVC system. To this end, the contact interface can be configured such that it permits etching of tissue; in the affixed state of the eye, the eye comes into contact with the contact interface only outside of the region in which an etching is intended to take place.

It is understood that the features mentioned above and the features still to be explained below can be used not only in the specified combinations but also in other combinations or on their own without departing from the scope of the present invention.

### BRIEF DESCRIPTION OF THE DRAWINGS

The invention is explained in greater detail below for example with reference to the accompanying drawings, which also disclose features essential to the invention. In the drawing:

FIG. 1 is a schematic representation of the geometry on the eye when the patient fixates in a wrong direction;

FIGS. 2a, 2b and 2c are schematic representations of the ablation geometry for UVL-LVC systems according to the prior art (2a and 2b) and for a focusing optical unit according to the invention (2c);

FIG. 3 depicts an example embodiment of a focusing optical unit;

FIG. 4 is a schematic representation for determining an effective corneal radius;

FIG. 5 is a schematic representation for elucidating the alignment of the focusing optical unit according to the invention with respect to the eye;

FIG. 6 is a schematic representation of the principle of geometry losses;

FIG. 7 is a graphical representation of normalized fluence losses for a system according to the prior art and for a focusing optical unit according to the invention;

FIG. 8 is a graphical representation of the percentage deviation of a normalized effective etch rate from a normalized target etch rate for different model approaches;

FIG. 9 depicts a basic arrangement of the optical beam path in an exemplary embodiment of a UVL-LVC system;

FIG. 10 depicts a basic arrangement of the optical beam path in a variant of a UVL-LVC system; and

FIG. 11 is a schematic representation of a UVL-LVC system.

### DETAILED DESCRIPTION

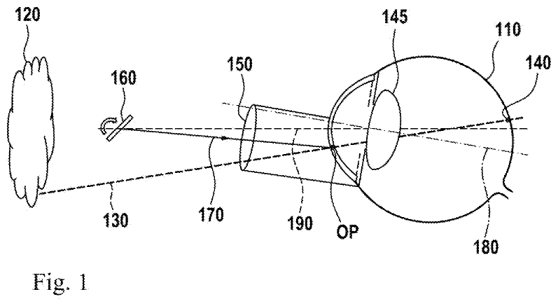

FIG. 1 shows a schematic representation of the geometry on the eye 110 when the patient fixates in a “wrong” direction. In the example shown, the eye 110 of the patient is not gazing at the center of a fixation cloud 120. In this case, an ablation profile 150 is not applied correctly along the necessary treatment axis, for example along a visual axis 130. The visual axis 130 is defined by the ophthalmic pole OP and the fixation of the patient. Hence, the ablation profile 150 is not applied at right angles to the visual axis 130. The relationships are depicted with much exaggeration in FIG. 1. FIG. 1 furthermore shows a fovea 140 of the eye, a crystalline lens 145, a scanner 160 (rotatable, represented by the bent arrow) of the UVL-LVC system for the lateral deflection of laser radiation 170, an axis of symmetry 180 (e.g., optical axis) of the eye 110 and an optical axis 190 of the UVL-LVC system.

An ablation profile 150 is not applied at the correct angle (i.e., with the center not on the surface normal, that is to say perpendicular to the visual axis 130) as a result of the “wrong” fixation of the patient's eye 110. This may occur if the patient preferably fixates in a largely fixed, but “wrong” direction, that is to say for example permanently gazes in a fixed direction that does not correspond to the center of the “fixation cloud” 120. This may occur should the patient, depending on refraction deficit and treatment duration, no longer be able to see the fixation target in focus during the operation. A prismatic correction error (tip/tilt) arises on account of the wrong fixation.

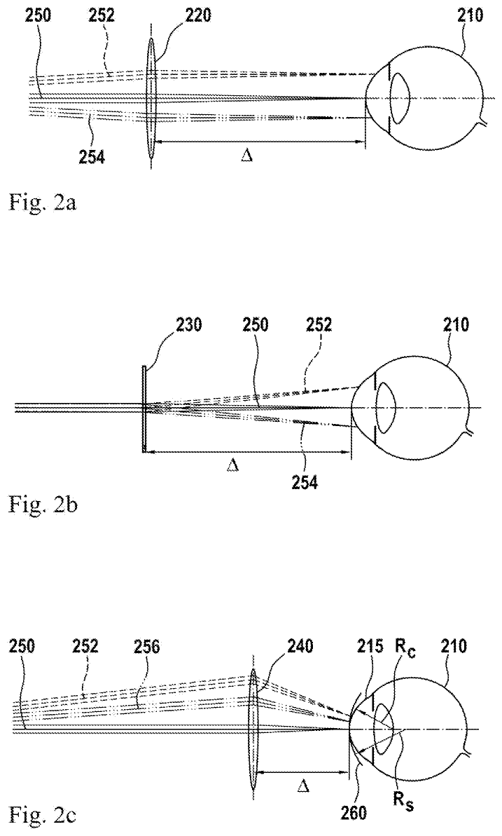

FIGS. 2a and 2b represent ablation geometries for optical systems according to the prior art. In this case, FIG. 2a shows the ablation geometry for a first optical system of a UVL-LVC system according to the prior art, which is characterized in that there is telecentric focusing of the ablation pulses. Beams 250, 252, 254 for a central position in a work surface, for a first edge position and for a second edge position, respectively, are shown. In this case, the beams 250, 252, 254 of the laser radiation are incident on a lens 220 of the focusing optical unit according to the prior art and are focused at (or near to) the corresponding positions on the eye 210 by this lens 220. In this case, the eye 210 is at a working distance Δ from the lens 220. The focusing optical unit according to the prior art shown here is telecentric on the image side (on the side of the eye), that is to say the chief rays of the beams 250, 252, 254 run in parallel between lens 220 and eye 210.

FIG. 2b shows the ablation geometry for a second optical system of a UVL-LVC system according to the prior art, which is characterized in that there is divergent focusing of the ablation pulses. Convergent laser light incident on a scanner 230 is shown, said laser light—depending on the scanner position—being focused at different positions on (or near to) the eye 210. This is shown for three positions with beams 250, 252, 254 for a central position in the work surface, for a first edge position in the work surface and for a second edge position in the work surface, respectively. The beams 250, 252, 254 are aligned divergently with respect to one another (with respect to the UVL-LVC system) between scanner 230 and eye 210. The eye 210 is at a working distance Δ from the scanner 230.

FIG. 2c shows the principle of the ablation geometry for a first variant of the focusing optical unit according to the invention. The latter is characterized in that convergent focusing of the ablation pulses is provided. The beams 250, 252, 256 are incident on the first lens arrangement 240 of the focusing optical unit when diverging from one another; in the process, the individual beams 250, 252, 256 are intrinsically parallel. As a result of the first lens arrangement 240, the beams 250, 252, 256 are directed convergently to one another in the direction of the focal area 260 and are in each case focused there. In this case, beam 250 corresponds to a central position in the focal field 260, beam 252 corresponds to an edge position in the focal field 260 and beam 256 corresponds to a position in the focal field 260 that is between the central position and the edge position. The schematically shown lens arrangement 240 contains further imaging elements and is only represented by an individual lens for the purposes of clarifying the principle.

Furthermore, the focal area 260 has a radius of curvature R_{S} in the example shown. This radius of curvature has the same sign as the corneal curvature with the radius of curvature R_{C} (“scanning radius of curvature”). Additionally, the two radii of curvature R_{C} and R_{S} have almost the same magnitude such that the focal area 260 extends close to the cornea 215.

The focusing optical unit shown in FIG. 2c likewise corresponds to a second variant of the focusing optical unit according to the invention. Here, the curved surface is identical to the curved focal area 260 in this example; the radius R_{F} of the surface curvature is identical in this case to the radius of the focal field curvature R_{S}. The chief rays of the beams 250, 254, 256 are directed at an angle to the curved surface by the focusing optical unit, the angle being less than 10° in relation to the surface normal at the location of impingement by the laser radiation. This is realized by way of the second lens arrangement 240.

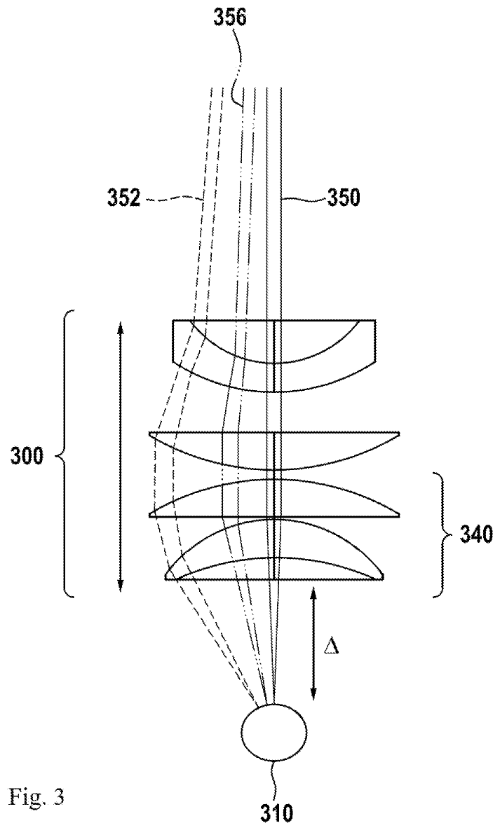

In FIG. 3, a lens section is depicted for an exemplary embodiment of a focusing optical unit 300 (in accordance with both variants), which is formed from spherical lenses. Laser radiation enters the focusing optical unit 300 from the side facing away from the eye 310. Plotted here are three (intrinsically approximately parallel) beams 350, 352, 356 for a central position on the eye 310, an edge position and a position between the central position and the edge position, respectively. These beams 350, 352, 356 are diverging from one another when incident on the focusing optical unit 300. In the example, the divergence is provided by way of a scanner (not plotted). The first lens arrangement 340 of the (first variant of the) focusing optical unit 300 provides the convergent focal field (not plotted) at a working distance Δ. In this exemplary embodiment, the focal field is curved (with a radius R_{S}=R_{C}=12 mm). In the example shown, the first lens arrangement 340 is identical to the second lens arrangement (according to the second variant) and the curved surface is also identical to the focal field (with R_{F}=R_{S}). The angle of incidence of the chief rays deviates by less than 10° from a perpendicular incidence on the curved surface for all beams 350, 352, 356.

The focusing optical unit shown here is particularly compact and has an installation size (length) of 54 mm in the case of an optical diameter of 56 mm, and provides a working distance of Δ=30 mm in the process.

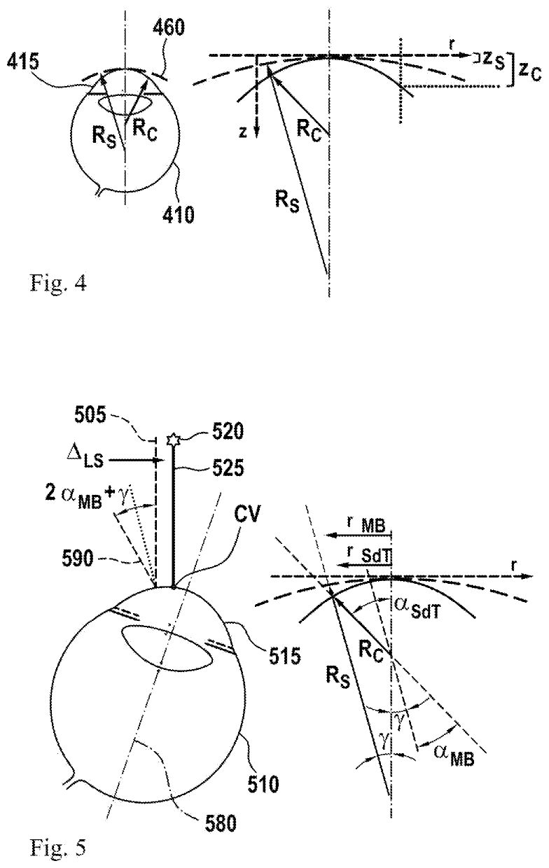

FIG. 4 shows a schematic illustration for determining an effective corneal radius for a curved focal field. Shown is an eye 410 having a cornea 415 with a corneal radius of curvature R_{C}. The focal field 460 provided by the focusing optical unit (according to the first variant; not depicted here) has a focal field radius of curvature R_{S} (“scanning radius of curvature”), where R_{S}>R_{C} applies.

According to the sphere model, z(R,r)=r²/(R+\sqrt{R²−r²}) applies. In this case, R describes the radius of a sphere, z describes the heights with respect to a tangent to this sphere, and r describes the distance along the tangent from the point of contact between circle and tangent.

The right-hand side of FIG. 4 shows the geometric conditions once again, in magnified fashion. For a difference height Δz, the following applies:

Δz(R_{C}, R_{S}, r)=z(R_{C}, r)−z(R_{S}, r)=z(R_{Δ}, r)

In this case, z_{C}:=z(R_{C}, r) and z_{S}:=z(R_{S}, r). This difference height Δz is intended to be determined by a difference radius of curvature R_{Δ}. This corresponds to an “effective” corneal curvature for light incident from the z-direction, for example like for solutions according to the prior art (see also FIG. 2a). As a result of the difference calculation, the cornea is “bent” upward—figuratively speaking—by the focal field radius of curvature, then allowing the calculation of the fluence loss to be carried out in a simplified manner, that is to say on the basis of an “effective” corneal curvature.

Using the aforementioned formula for a sphere model, the following arises from the equation for the difference height Δz:

$$\frac{1}{{R}_{Δ} + \sqrt{{R}_{Δ}^{2} - {r}^{2}}} = \frac{1}{{R}_{C} + \sqrt{{R}_{C}^{2} - {r}^{2}}} - \frac{1}{{R}_{S} + \sqrt{{R}_{S}^{2} - {r}^{2}}}$$

This relationship for determining R_{Δ} applies to all r (in particular to r=0). This results in:

$$\frac{1}{{R}_{Δ}} = \frac{1}{{R}_{C}} - \frac{1}{{R}_{S}}$$

Consequently, the following arises for the “effective” corneal radius of curvature:

$${R}_{Δ} = \frac{{R}_{C} \cdot {R}_{S}}{{R}_{S} - {R}_{C}}$$

Consequently, with a typical corneal radius of curvature of R_{C}=7.86 mm, values of approximately R_{Δ}≈450 mm to approximately R_{Δ}≈15 mm arise for the effective corneal curvature for focal field radii of curvature with values between approximately 8 mm and approximately 16 mm. In these regions the advantage of an improved fluence loss function is particularly clear since these effective radii are significantly larger than the typical radius of curvature of the cornea, and so an impingement of the cornea with laser light is significantly closer to perpendicular incidence than in the prior art.

Even though the observations shown here were made for a first variant of the focusing optical unit, they also apply to a focusing optical unit according to the second variant. In this case, the curved surface is identical to the focal field 460 (with R_{F}=R_{S}).

FIG. 5 depicts a schematic illustration for clarifying the effect of decentrations of the focusing optical unit (according to the first variant) according to the invention with respect to the eye. To demonstrate this, a non-coaxial alignment of a patient when fixating a fixation target 520 is shown to the left in FIG. 5. Although the target 250 is fixated (it should be observed that a collimated beam 525 from the target 520 is used in this example), the patient is not aligned coaxially with the optical system. A correct centration according to the CSCLR condition would correspond to an alignment of the eye 510 with respect to the directed (collimated) beam 525 of the target 520 with Δ_{LS}=0 mm (aligned with the “corneal vertex” CV). The example of FIG. 5 plots a shift of a system axis 505 (optical axis) of the focusing optical unit according to the invention of Δ_{LS}=2 mm with respect to the directed laser beam 525 for a centration in accordance with the CSCLR condition. Furthermore, a focal field radius of curvature R_{S} of 12 mm was assumed. In this example, the system axis 505 arises by way of a ray guided by the scanners of the UVL-LVC system (not plotted), for example by way of an alignment beam in the zero position of the scanners. The geometric considerations plotted to the right in FIG. 5 serve for the estimation of the angle of incidence α_{MB} of a laser ray along the system axis 505 of the focusing optical unit on the cornea 515 for a focusing optical unit according to the invention, as a function of the pupil coordinate r_{MB}≈r_{SdT}, where r_{MB} and r_{SdT} describe the shift of the system axis 505 with respect to the centration in accordance with the CSCLR condition for a focusing optical unit according to the invention and a focusing optical unit according to the prior art, respectively. The following holds true:

$$γ = {sin}^{- 1} ( \frac{{r}_{M B}}{{R}_{S}} ) \approx {sin}^{- 1} ( \frac{{r}_{S d T}}{{R}_{S}} )$$

In this case, γ is the angle between an incident laser ray for the focusing optical unit according to the invention with a convergent and curved focal field with respect to a focusing optical unit according to the prior art with telecentric focusing on a plane focal field. The following arises for the angle of incidence α_{SdT} according to the prior art:

$${α}_{S d T} = {sin}^{- 1} ( \frac{{r}_{S d T}}{{R}_{C}} )$$

By contrast, the following applies to the focusing optical unit according to the invention:

α_{MB}≈α_{SdT}−γ

Then, the angle of reflection 2α_{MB}+γ arises for the reflected laser ray 590 in the case of a lateral displacement Δ_{LS}. In this case, Δ_{LS} is used in the calculation for the pupil coordinate r_{SdT}(≈r_{MB}). In this case, the ophthalmic pole (OP) and the corneal vertex (CV) are equated to one another WLOG. In this example, a reflection angle of approximately 2α_{MB}+γ=20° arises for an offset of Δ_{LS}=2 mm. This angle is detected without problems by the focusing optical unit and can be processed in the UVL-LVC system.

The axis of symmetry 580 of the eye 510 is also depicted in FIG. 5. Attention is drawn to the fact that the representation is simplified in relation to the corneal shape.

Even though the observations shown here were made for a first variant of the focusing optical unit, they also apply to a focusing optical unit according to the second variant. In this case, the curved surface is identical to the curved focal field (with R_{F}=R_{S}).

In the discussed configuration, the focusing optical unit (in both variants) is therefore particularly well suited to identify reflections such as the first Purkinje image and/or the vertex, and hence advantageously allows an improvement in the centration of the patient's eye with respect to the UVL-LVC system.

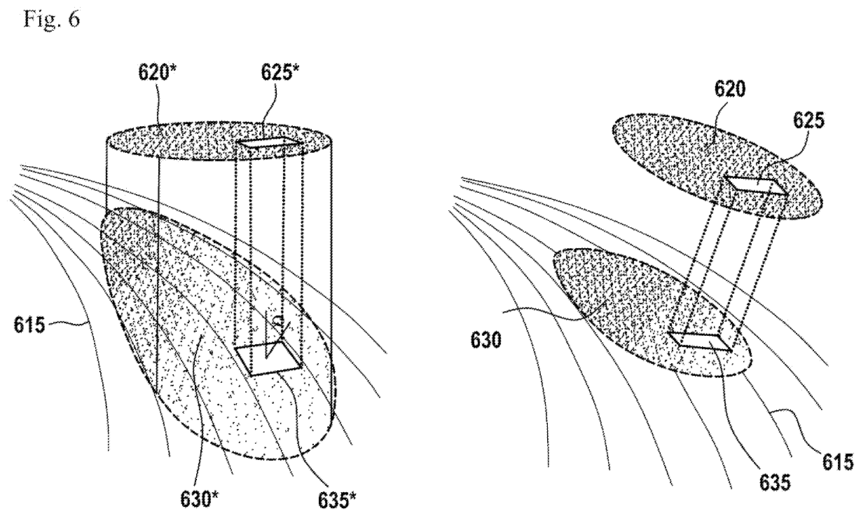

FIG. 6 shows a schematic representation of the principle of geometry losses. In this case, the geometry for the prior art is shown to the left in FIG. 6 (reference signs are marked by an asterisk “*”) while the right-hand side depicts the conditions for a focusing optical unit (according to both variants) according to the invention (with reference signs without an asterisk). The pulse ablation shape 620, 620* (corresponds to the ablation-effective fluence distribution of the radiated-in ablation laser pulse on a plane perpendicular to the direction of incidence) is deformed by the geometry of the irradiation on the cornea 615 to form the projected pulse ablation shape 630, 630* (pulse ablation footprint on cornea). For a geometry according to the prior art, this also changes the fluence distribution on the cornea 615 with respect to the radiated-in pulse ablation shape 620*. This is made clear by the hatching on the projected pulse ablation shape 630*. In this case, a constant fluence distribution was assumed for the pulse ablation shape 620, 620* for a better visualization. For the geometry in the case of the focusing optical unit according to the invention, the shape of the radiated-in pulse ablation shape 620 largely corresponds to the shape of the projected pulse ablation shape 630; as a result, the fluence distribution in the projected pulse ablation shape 630 remains largely constant. This is made possible by the convergent focal field according to the invention or by the perpendicular incidence according to the invention on the curved surface.

In UVL-LVC systems, laser pulses are typically designed approximately as low-order supergaussians, from which the pulse ablation shape 620, 620* can then be calculated. By way of example, the latter can be implemented from a given pulse shape with the aid of the blow-off model. The geometry loss is modeled as a cosine function for an infinitesimal surface element dA 625, 625*. The following applies: cos(α)=dA/dA′, with the angle of incidence a (angle of incidence with respect to the local surface normal on the cornea 615) and the projected infinitesimal surface element dA′635, 635*. In comparison with solutions according to the prior art (to the left in FIG. 6), the geometry losses are reduced for the focusing optical unit according to the invention (to the right in FIG. 6) on account of the small angle of incidence α. Moreover, the pulse ablation footprint on cornea 635 only deviates slightly from the radiated-in pulse ablation shape 625.

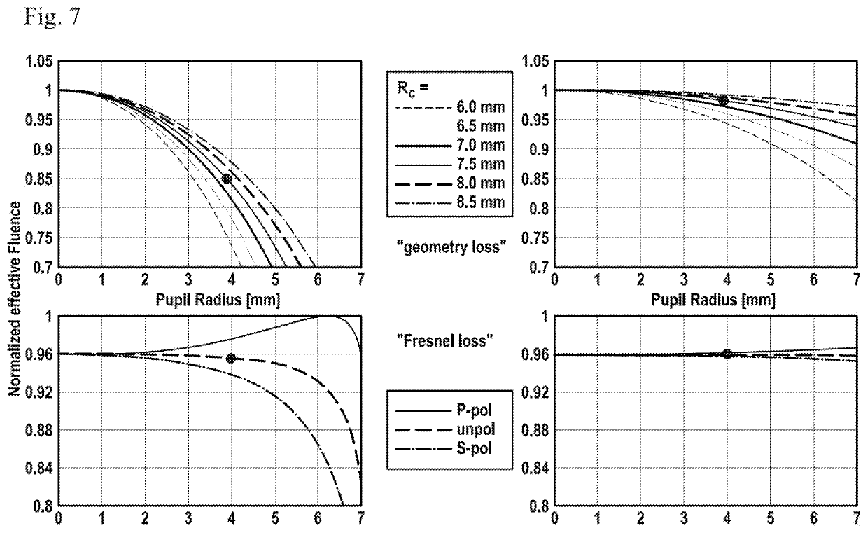

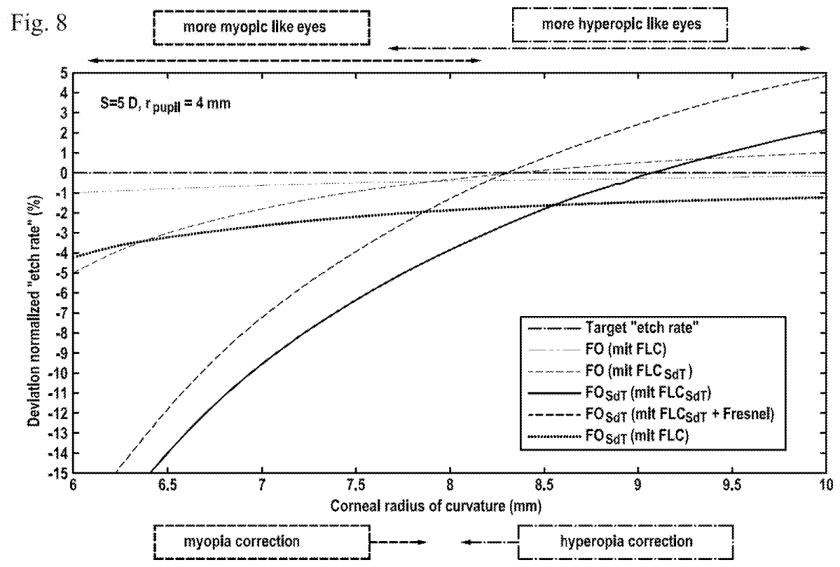

FIG. 7 shows normalized fluence losses for a system according to the prior art (left-hand side) according to an arrangement as depicted in FIG. 2b and for a focusing optical unit according to the invention (right-hand side, first variant). A focal field curvature with a radius R_{S} of 12 mm and a working distance Δ of 40 mm was assumed for the focusing optical unit according to the invention. The plotted points mark typical work points during the ablation for a typical corneal curvature of R_{C}=7.5 mm and in the case of a pupil radius of 4 mm (transition from the optical zone to the transition zone).

The two upper graphs in FIG. 7 show the normalized fluence losses (“Normalized effective Fluence”) as a function of a pupil radius (“Pupil Radius”) in millimeters for different radii of curvature of the cornea from R_{C}=6 mm to 8.5 mm (in increments of 0.5 mm) on account of geometry losses (“geometry loss”). It is possible to identify that the geometry losses are significant for solutions according to the prior art. They are approximately 15% at the chosen work point. By contrast, the corresponding losses for the focusing optical unit according to the invention are only approximately 1%. Moreover, a clear dependence on the pupil coordinate (pupil radius) is evident in the prior art. The losses increase significantly in the outer regions of the pupil.

The two lower graphs in FIG. 7 show the normalized fluence losses (“Normalized effective Fluence”) as a function of a pupil radius (“Pupil Radius”) in millimeters on account of Fresnel losses (“Fresnel loss”). It is possible to identify that, apart from a constant component of approximately 4 percent, the Fresnel losses for unpolarized light are only a small component of a variation over the pupil (in the prior art, left) and have practically no influence in the solution according to the invention (right). However, there is a significant dependence on the pupil radius in the case of polarized light in the prior art. This disadvantage is overcome for the solution according to the prior art. The calculations were carried out using a refractive index of approximately 1.5 for the stroma; the calculations provide no fundamentally different results in the case of a more realistic value of n=1.377 for the stroma. The constant component would reduce to approximately 3% therewith.

Even though the observations shown here were made for a first variant of the focusing optical unit, they also apply to a focusing optical unit according to the second variant. In this case, the curved surface is identical to the curved focal field (with R_{F}=R_{S}).

FIG. 8 graphically depicts the percentage deviation of a normalized effective etch rate from the normalized target etch rate (target ablation, also referred to as “Deviation normalized etch rate”; specifications in percent) for a focusing optical unit according to the prior art (FO_{SdT}) and a focusing optical unit according to the invention (FO, first variant) for different model approaches for a laser pulse fluence loss compensation function (FLC: fluence loss compensation) as a function of the (preoperative) radius of curvature of the cornea or of the stroma (“Corneal radius of curvature (mm)”). In this case, the shown calculations resort to the results of the explanations in relation to the geometry and Fresnel losses. The influence thereof and the differences in an energy correction for a solution according to the prior art (as shown in FIG. 2b) and a solution using the focusing optical unit according to the invention were examined on the basis of model calculations. An effective ablation rate was calculated on the basis of these calculations. The modeling is based on a simplified spherical corneal model, spherical corrections and the “blow-off” model, to simplify the understanding.

The calculations apply to the laser pulse maximum fluence (“peak fluence”). A spatial extent of the laser pulse (or of the laser radiation) was not taken into account for the calculation of the geometry and Fresnel losses. This is an approximation which improves as the pulse diameter decreases (ratio of pulse diameter to the diameter of the optical zone). Attention is drawn to the fact that taking account of the relationships shown below once again benefits the solution according to the invention. As representative values in all cases, 160 mJ/cm² was taken for the “peak fluence” (F₀) and 48 mJ/cm² was taken for the stroma threshold ablation fluence (“threshold fluence”). The refractive index of the stroma was taken to be n=1.377.

FIG. 8 depicts the results of the modelings. The percentage deviation of the “etch rate” from the target etch rate as a function of the preoperative corneal radius of curvature (“Corneal radius of curvature”) is considered. The deviation of the target etch rate (Target “etch rate”) itself is naturally zero, which defines the zero line.

A spherical 5 D (dpt) correction in the case of a 4 mm pupil radius was considered as a case study. Attention is drawn to the fact that optical zones are usually located up to 6 mm, and often therebeyond, and, with transition zones of 1.5 mm, even lead to pupil radius coordinates closer to 4.5 mm (9 mm overall ablation diameter). Typically, hyperopic eyes (labeled as “more hyperopic like eyes”) exhibit on average larger corneal radii of curvature and require steepening, that is to say a reduction, of the corneal radii of curvature for correcting the visual defect (labeled by “hyperopia correction”). The opposite applies to myopic eyes (labeled “more myopic like eyes” and “myopia correction”).

The curves in the diagram of FIG. 8 arise as a result of the fact that the difference in the fluence loss-compensated etch rates from preoperative to postoperative is determined on the basis of the radii of curvature (given preoperatively, calculated postoperatively on the basis of the spherical correction). In this case, the actual fluence loss for the preoperative and postoperative states is initially calculated on the basis of the respective radii of curvature (“physically correctly”). Then the compensation of the loss is calculated. This is based on the preoperative radius of curvature (UVL-LVC system with a focusing optical unit according to the invention, labeled as “FO (with FLC)”) or on a fixed radius of curvature of 7.86 mm (labeled as “FO_{SdT} (with FLC_{SdT})” for a UVL-LVC system with a focusing optical unit according to the prior art (FO_{SdT})).

The line labeled “FO_{SdT} (with FLC_{SdT})” represents the deviation of the effective etch rate from the normalized target etch rate for a UVL-LVC system with a fluence loss compensation according to the prior art. This emerges from the calculated loss function and a typical compensation function according to the prior art, which does not consider the Fresnel losses but contains the cosine-dependent projection of the surface elements (cos(a) in FIG. 6) on the basis of a fixed corneal radius of curvature R_{C} of 7.86 mm.

The line labeled “FO_{SdT} (with FLC_{sdT}+Fresnel)” represents the deviation for a system according to the prior art with a fluence loss compensation according to the prior art if the Fresnel losses are additionally considered. Essentially, it is possible to identify a shift of the function to the left, that is to say to smaller corneal radii of curvature (or “upward”, depending on the point of view). This is due to the fact that the Fresnel losses for unpolarized excimer laser pulses vary only slightly with the angle of incidence (cf. work point in FIG. 7).

The line labeled “FO (with FLC)” represents the deviation of the effective etch rate from the normalized target etch rate for a UVL-LVC system with a focusing optical unit according to the invention (FO) and a compensation according to the invention (FLC). The latter considers the optical geometry (in this case, focal field curvatures R_{S} of 12 mm and a working distance Δ of 40 mm) of the focusing optical unit according to the invention for the compensation function. The curve once again arises from the calculated loss function and the compensation function according to the invention. The latter also considers the Fresnel losses in addition to the geometry losses (which are low in comparison with FO_{SdT}) and uses the preoperative corneal radius of curvature for the compensation. The variation of this function over the corneal radii of curvature is significantly reduced in relation to a focusing optical unit according to the prior art. This leads to a significantly improved predictability or to a reduction in the variation of the refractive result, as will still be explained below.

The line labeled “FO (with FLC_{SdT})” represents the deviation of the effective etch rate from the normalized target etch rate for a UVL-LVC system with a focusing optical unit according to the invention (FO), which arises if use were to be made of the above-described compensation function according to the prior art (FLC_{SdT}). Even in this case, the deviations of the effective etch rates for the arrangement according to the invention would still be approximately one order of magnitude smaller than in the case of the prior art. This is substantially based on the above-described “more good-natured” curve of the geometry losses over the pupil coordinates (see FIG. 7). An improved variant of this compensation (which would consider the accumulation points of the radii of curvature for hyperopic and myopic eyes) could for example be used by physicians who do not determine preoperative keratometry or do not use the latter.

Finally, the line labeled “FO_{SdT} (with FLC)” represents the deviation of the effective etch rate from the normalized target etch rate for a UVL-LVC system with a focusing optical unit according to the prior art (FO_{SdT}) if the compensation function according to the invention FLC were to be applied. The profile of the curve shows that the compensation function according to the invention FLC already leads to significant improvement in the predictability of the refractive result.

Even though the observations shown here were made for a first variant of the focusing optical unit, they also apply to a focusing optical unit according to the second variant. In this case, the curved surface is identical to the curved focal field (with R_{F}=R_{S}).

The intention is now to explain why the concept according to the invention is particularly advantageous in relation to an improved predictability of the refractive results.

The curve of the percentage deviations of the etch rates for a UVL-LVC system with a focusing optical unit according to the prior art, shown in FIG. 8, could for example be compensated by nomograms. As a rule, nomograms are created by a linear regression of the refractive correction results (attempted vs. achieved) in comparison with the correction target, and are intended to minimize these differences. As a result, the nomograms do not contain any dependence on the preoperative keratometry (“K-values”), and a mean keratometry value for which the nomogram correction is optimal arises. If there now is a treatment of a patient with a keratometry that deviates from this mean value, the obtained nomogram correction does not fit perfectly to the corresponding etch rate deviation (to be compensated by the nomogram). That is to say, a non-optimal compensation function is applied.

For prior art systems, the keratometry can now be taken into account in the nomogram as follows: Let the mean value of the keratometry in the considered hyperopia group for the nomogram correction be R_{C}=8.25 mm. If a hyperopic patient with a keratometry deviating therefrom and assumed to be R_{C}=7.25 mm is now treated, it is possible to read the difference in the ablation rate that would not be corrected from the diagram in FIG. 8, simply as the difference in the deviation (ordinate) in percent between these two radii of curvature. A miscorrection of approximately 5% (for a 5 diopter sphere correction in the case of a pupil radius of 4 mm) would arise for this case in solutions according to the prior art. Thus, the peripheral pupil regions of the correction ablation profile and the transition zone, in particular, would be represented incorrectly. In the case of assumed 14 μm (maximum etch depth) per diopter, this would correspond to approximately 3.5 μm. This deviation is presented both as a spherical aberration and a refractive miscorrection of approximately 0.25 dpt. Even though this case initially appears hypothetical, it nevertheless is not unrealistic and would become apparent as a deviation in the prediction in “attempted vs. achieved” diagrams.

The non-optimal energy compensation, which quite fundamentally is due to the ablation geometry of FIGS. 2a and 2b of laser systems according to the prior art, thus leads to a broader variation in the prediction of the refractive results, especially in conjunction with nomogram correction.

One could object that it is possible to apply an exact energy correction to UVL-LVC systems according to the prior art. This is fundamentally true but not implementable in practice. Ideally, the current corneal shape at the pupil position during the ablation would be determined for the next laser pulse. However, this cannot be done with current technology (processing speed, control speed), and would be accompanied by other limitations and problems. Alternatively, attempts could be made to take account of the current corneal shape during the pulse sorting process. This would represent a great advance and is categorically doable from the view of the sorting algorithms (sorting of the pulses for etch optimization). However, a subsequent and necessary thermal sort would then be impossible without undoing the previously considered improvement (resorting of the pulses). There is currently no prospect of a physical and mathematical method for combining sort and thermal sort (“simultaneously”). Therefore, the minimizing of the energy correction by the optical unit according to the invention and the consideration of the K-values offers an improvement of the predictability of the results, a reduction in the variation and, moreover, also an improvement of the nomograms as these have to correct smaller deviations per se.

A feature of the planning unit according to the invention is that the remaining aberrations and hence the spot variations in the focal field are measured or physically modeled for the calculation of the planning data and are made available to the sorting algorithm. These data can be used to determine the accurate ablation-effective fluence distribution of the pulses in the focal field as a function of the focal field position, and hence to take this into account when sorting the pulses (see FIG. 6).

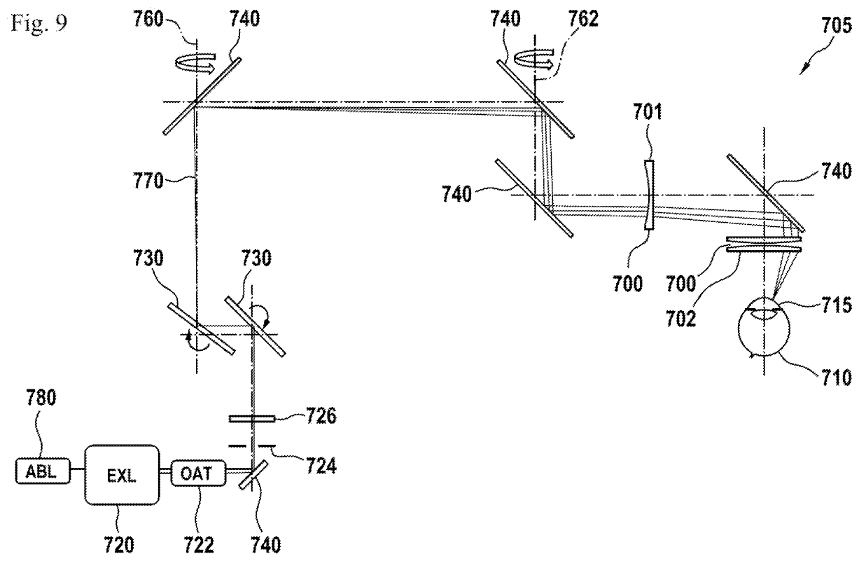

FIG. 9 shows a basic arrangement of the optical beam path in an exemplary embodiment of a UVL-LVC system 705. Laser radiation 770 is provided by an excimer laser 720 as a UV laser source. The laser radiation 770 is attenuated by an (optional) optical attenuator 722, deflected by a deflector 740, is incident on a stop (or a pinhole) 724 and subsequently reaches a beam shaper 726. The latter serves to shape the beam of the raw excimer laser beam into a Gaussian or supergaussian pulse fluence distribution. By way of scanners 730, the laser beam 770 can be deflected laterally in the x- and y-directions (depicted by way of bent arrows). From here, the laser radiation 770 is guided in a first articulated arm. In the exemplary embodiment shown, the latter is movably connected to a base unit (not plotted) by way of a first rotary joint 760 (symbolized by an axis of rotation and a rotation arrow). The base unit comprises the laser source 720, the optical attenuator 722, the stop 724 (and the deflector 740 which is situated in the beam path between the optical attenuator 722 and the stop 724), the beam shaper 726 and the scanners 730. The first articulated arm is movably connected to a second articulated arm by way of a second rotary joint 762 (symbolized by an axis of rotation and a rotation arrow) on the side distant from the base unit. The laser radiation 770 is guided into the second articulated arm via two further deflectors 740 by way of the second rotary joint 762. From there, the laser radiation 770 is directed in the direction of the eye 710 by way of a further deflector 740. In this case, the laser radiation 770 is focused on the cornea 715 of the eye 710 by way of a focusing optical unit 700 according to the invention (in both variants). In this case, the focusing optical unit 700 has a two-part structure. A deflector 740 is situated in the beam path between the first lens group 701 and the second lens group 702. The required lenses of the two lens groups 701, 702 are only depicted schematically and not physically correctly.

Furthermore, the UVL-LVC system comprises what is known as an “alignment beam laser” 780. The latter serves to adjust the optical system and/or carry out an alignment with respect to the eye. The laser beam of the alignment beam laser follows the laser beams 770 of the excimer laser 720 on the cornea 715 and its focus. The “alignment beam laser” 780 is situated in the base unit of the UVL-LVC system 705.

Attention should be drawn to the fact that one or more deflectors 740 may also be embodied as beam splitters. This allows integration of other components, for example detectors for the detection of the collected corneal reflections or an observation camera. Various arrangements immediately evident to an expert but not plotted in FIG. 9 are possible to this end.

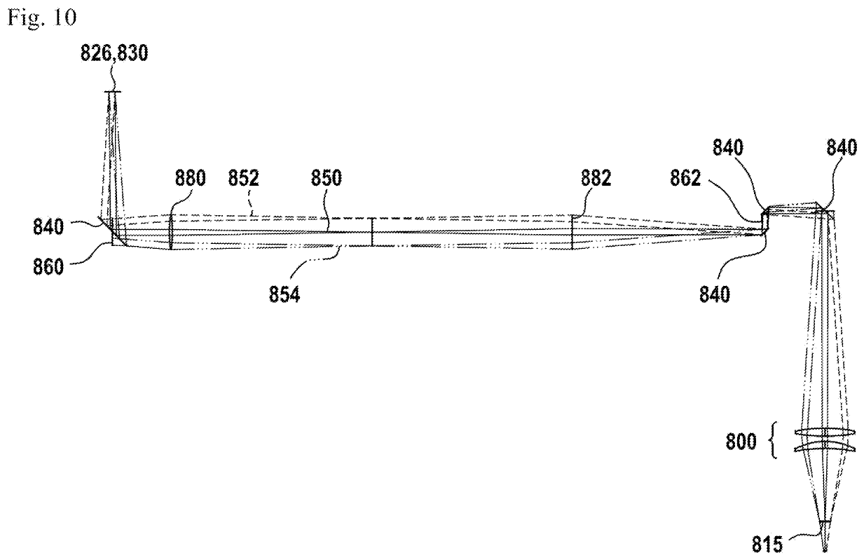

FIG. 10 shows a basic arrangement of the optical beam path in a variant of a UVL-LVC system. The laser radiation is shown here for three exemplary beams 850, 852, 854 from the beam shaper 826 and the scanner 830 (depicted together here for simplification purposes) to the cornea 815 via two articulated arms, which have deflectors 840 and are movably connected to the base unit (not depicted here) by a first rotary joint 860 and movably connected to one another by a second rotary joint 862, and via the focusing optical unit 800 (in both variants). In this case, the beams 850, 852, 854 correspond to three different locations on the cornea 815.

In contrast to the exemplary embodiment shown in FIG. 9, beam guidance is realized here by way of a relay optical unit. For simplification purposes, the relay optical unit is depicted here by way of a first relay lens 880 and a second relay lens 882. As a result, there is a larger beam diameter and an image inversion. Here, the option of lengthening the beam path is advantageous. Various other options arise for the imaging in the beam path (via the articulated arms). In this respect, the focusing optical unit 800 is uncritical.

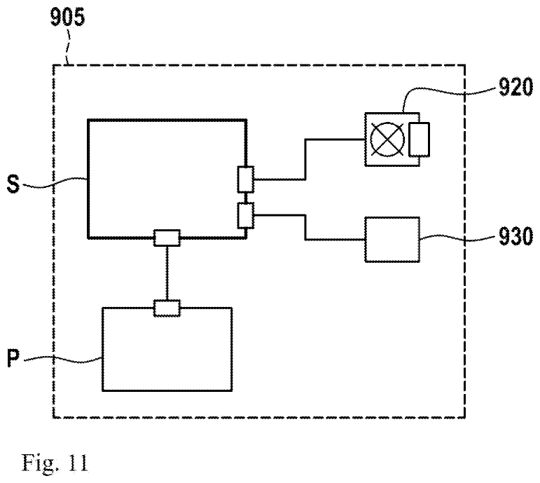

FIG. 11 shows a schematic illustration of a UVL-LVC system 905. The UVL-LVC system 905 comprises a UV laser source 920, a scanner 930, a control unit S and a planning unit P. For data exchange between the control unit S and the UV laser source 920, the scanner 930 and the planning unit P, the control unit S has interfaces (represented by boxes on the control unit S), by application of which the data line can be transferred by way of cables. The planning unit P likewise comprises an interface (depicted as a box on the planning unit P) for data exchange with the control unit S. A wireless transfer is likewise possible. The planning unit P has a computing unit (not depicted here), by operation of which the planning data are calculated.

The aforementioned features of the invention, which are explained in various exemplary embodiments, can be used not only in the combinations specified in an exemplary manner but also in other combinations or on their own, without departing from the scope of the present invention.

1.-13. (canceled)

14. A focusing optical unit for a UV laser-based system for vision correction (UVL-LVC system),
the UVL-LVC system having a UV laser source that provides laser radiation; and
a scanning system that laterally scans the laser radiation in x- and y-directions:
wherein the focusing optical unit is configured to focus the laser radiation in a focal field; and
wherein the focusing optical unit comprises a first lens arrangement which is configured to provide a convergent focal field.

15. The focusing optical unit as claimed in claim 14, wherein the scanning system that laterally scans the laser radiation in the x- and y-directions also scans the laser radiation in a z-direction.

16. The focusing optical unit as claimed in claim 14, wherein the convergent focal field has a focal field diameter selected from a group consisting of at least 6 mm, at least 8 mm and at least 10 mm.

17. The focusing optical unit as claimed in claim 14, wherein each location in the convergent focal field has a local center of curvature on a side facing away from the focusing optical unit.

18. The focusing optical unit as claimed in claim 17, wherein each location in the focal field has a focal field curvature selected from a group consisting of a radius RS ranging from 8 mm to 50 mm, from 10 mm to 30 mm and from 12 mm to 20 mm.

19. A focusing optical unit for a UV laser-based system for vision correction (UVL-LVC system), the focusing optical unit comprising:
a UV laser source that provides laser radiation; and
a scanning system that laterally scans the laser radiation in x- and y-directions;
wherein the focusing optical unit comprises a lens arrangement that provides perpendicular incidence of laser radiation on a curved surface, wherein each location on the curved surface has a local center of curvature on a side facing away from the focusing optical unit.

20. The focusing optical unit as claimed in claim 19, wherein the curved surface has a diameter selected from a group consisting of at least 6 mm, at least 8 mm and at least 10 mm.

21. The focusing optical unit as claimed in claim 19, wherein the curved surface has a surface curvature with a radius R_{F} selected from a group consisting of a range from 8 mm to 50 mm, a range from 10 mm to 30 mm and a range from12 mm to 20 mm.

22. The focusing optical unit as claimed in claim 14,
configured to have a working distance (Δ) in a range from 20 mm to 50 mm;
an optical aperture selected from a group consisting of greater than 40 mm, greater than 50 mm, and greater than or equal to 60 mm, or
a combination of the foregoing.

23. The focusing optical unit as claimed in claim 14, comprising a first lens which has a first lens material with a first refractive index and a first Abbe number and comprising a second lens which has a second lens material with a second refractive index and a second Abbe number, with the first refractive index differing from the second refractive index and the first Abbe number differing from the second Abbe number.

24. The focusing optical unit as claimed in claim 23, wherein the first lens has a negative optical power, the second lens has a positive optical power and the first refractive index is greater than the second refractive index.

25. The focusing optical unit as claimed in claim 14, wherein the focusing optical unit has at least two lens groups along a beam path with a non-imaging optical element being arranged therebetween.

26. A planning unit for generating planning data for a UV laser-based
system for vision correction (UVL-LVC system), the UVL-LVC system comprising:
a UV laser source that provides laser radiation;
a scanning system that laterally scans the laser radiation in x- and y-directions;
a focusing optical unit that directs the laser radiation to a work surface; and
a control unit that controls the UVL-LVC system that is configured to take planning data into consideration;
wherein the planning unit is configured to take account of:
geometry losses,
Fresnel losses,
a spatial extent of the laser radiation in the work surface or a combination of the foregoing when calculating the planning data; and
wherein the planning unit has an interface operably coupled to the control unit, by which the planning data can be made available to the control unit
wherein the focusing optical unit:
focuses the laser radiation in a focal field; and wherein the focusing optical unit further comprises a first lens arrangement which is configured to provide a convergent focal field; or
wherein the focusing optical unit comprises a second lens arrangement that provides perpendicular incidence of laser radiation on a curved surface, wherein each location on the curved surface has a local center of curvature on a side facing away from the focusing optical unit.

27. (canceled)

28. The planning unit as claimed in claim 26,
wherein the focusing optical unit includes the first lens arrangement configured to provide the convergent focal field; and
wherein the convergent focal field has a focal field diameter selected from a group consisting of at least 6 mm, at least 8 mm and at least 10 mm; or
wherein each location in the convergent focal field has a local center of curvature on a side facing away from the focusing optical unit; or
wherein each location in the focal field has a focal field curvature selected from a group consisting of a radius RS ranging from 8 mm to 50 mm, from 10 mm to 30 mm and from 12 mm to 20 mm.

29. (canceled)

30. (canceled)

31. (canceled)

32. The planning unit as claimed in claim 26,
wherein the focusing optical unit includes the second lens arrangement that provides perpendicular incidence of laser radiation on the curved surface; and
wherein the curved surface has a diameter selected from a group consisting of at least 6 mm, at least 8 mm and at least 10 mm; or
wherein the curved surface has a surface curvature with a radius R_{F} selected from a group consisting of a range from 8 mm to 50 mm, a range from 10 mm to 30 mm and a range from12 mm to 20 mm; or
wherein the planning unit has a working distance (A) in a range from 20 mm to 50 mm, or
wherein the planning unit has an optical aperture selected from a group consisting of greater than 40 mm, greater than 50 mm, and greater than or equal to 60 mm; or
wherein the planning unit comprises a first lens which has a first lens material with a first refractive index and a first Abbe number and comprising a second lens which has a second lens material with a second refractive index and a second Abbe number, with the first refractive index differing from the second refractive index and the first Abbe number differing from the second Abbe number; or
wherein the focusing optical unit has at least two lens groups along a beam path with a non-imaging optical element being arranged therebetween; or
a combination of the foregoing.

33. (canceled)

34. (canceled)

35. (canceled)

36. (canceled)

37. (canceled)

38. A UV laser-based system for vision correction (UVL-LVC system), comprising
a focusing optical unit for a UV laser-based system for vision correction (UVL-LVC system) as claimed in claim 14; and
a planning unit for generating planning data for a UV laser-based system for vision correction (UVL-LVC system) comprising:
a control unit that controls the UVL-LVC system that is configured to take planning data into consideration;
wherein the planning unit is configured to take account of
geometry losses,
Fresnel losses,
a spatial extent of the laser radiation in the work surface or a combination of the foregoing when calculating the planning data; and
wherein the planning unit has an interface operably coupled to the control unit, by which the planning data can be made available to the control unit.

39. The focusing optical unit as claimed in claim 14, wherein the focusing optical unit is configured to produce round laser spots having a full width at half maximum (FWHM) in a range from 0.3 to 0.8 mm in the focal field with a deviation chosen from a group consisting of less than 20% and less than 10%.

40. The focusing optical unit as claimed in claim 14, wherein the first lens arrangement which is configured to provide a convergent focal field is further configured to take into account a difference radius of curvature (R_{Δ}) which represents an effective corneal curvature having effective corneal curvature values ranging from RA of 15 mm to R_{Δ} of 450 mm for focal field radii of curvature of between 8 mm and 16 mm thereby facilitating an improved fluence loss function.

41. The focusing optical unit as claimed in claim 19, wherein the lens arrangement that provides perpendicular incidence of laser radiation on the curved surface is further configured such that the incidence of the laser radiation on the curved surface has an angle with a normal to the curved surface at a location of incidence that is selected from a group consisting of no more than 10 degrees, no more than five degrees and no more than 2 degrees.

42. The focusing optical unit as claimed in claim 19, wherein the lens arrangement that provides perpendicular incidence of laser radiation on the curved surface is further configured such that the incidence of the laser radiation on the curved surface occurs at a spot size in a range selected from a group consisting of between 0.3 mm to 1.5 mm and between 0.5 mm to 1.0 mm.
